(19) United States
(12) Patent Application Publication
(10) Pub. No.: US 2015/0190280 A1
(43) Pub. Date: Jul. 9, 2015

(54) Irradiation Method and Apparatus

(71) Applicant: Lions Eye Institute Limited, Nedlands (AU)

(72) Inventors: Dao-Yi Yu, Nedlands (AU); Stephen John Cringle, Nedlands (AU)

(73) Assignee: Lions Eye Institute Limited, Nedlands (AU)

(21) Appl. No.: 14/412,904

(22) PCT Filed: Jul. 11, 2013
(86) PCT No.: PCT/AU2013/000771

§ 371 Date: Jan. 5, 2015

(30) Foreign Application Priority Data
Jul. 16, 2012 (AU) 2012903038

(51) Int. Cl. A61F 9/008; A61B 18/22; G01N 21/33

(52) U.S. Cl. CPC A61F 9/00802; G01N 21/33; A61B 18/22; A61B 2018/00642

(57) ABSTRACT

An apparatus for irradiating a specimen: that includes an optical transmitter for transmitting light from a laser source; an optical probe configured to receive the light from the optical transmitter and to apply the light upon emission from an optical exit to the specimen; a position detector adapted to detect a position of the optical probe in a longitudinal direction and to output a signal indicative of the position or of a change in the position relative to a surface forward of the optical probe; a drive coupled to the optical probe and adapted to controllably adjust a position of the optical probe in the longitudinal direction; and a feedback controller adapted to receive the signal from the position detector and to control the drive to control the position to keep the optical probe at substantially a constant position relative to the surface forward of the optical probe.

### FIELD OF THE INVENTION

The present invention relates to an irradiation method and apparatus, of particular but by no means exclusive application in ablating tissue, and especially soft tissue (such as the retina, vessel wall, trabecular meshwork, or other tissue), in a liquid environment.

### BACKGROUND OF THE INVENTION

Infrared sources, such as CO2, erbium-YAG and holmium:YAG lasers, have undergone trials, involving optical fiber delivery to a surgical target. Though adapted for use in intraocular surgery, problems include collateral, thermal damage to surrounding tissue and shock-wave effects.

UV lasers are widely accepted for use in corneal refractive surgery, such as photorefractive keratectomy (PRK) and laser intrastroma keratomileusis (LASIK), and provide good control of ablation depth and minimal damage to surrounding tissue. However, such systems are adapted for use in gaseous environments—that is, typically the atmosphere.

UV lasers at 266 nm have been extensively studied for use in tissue ablation in liquid environments; they are closely matched to the absorption peak of proteins in some target tissues and afford good control of ablation depth with minimal damage to surrounding tissue.

UV lasers at 213 nm have also been extensively studied for use in tissue ablation in liquid environments. They allow good control of ablation depth and minimal damage to surrounding tissue, but provide poor penetration in liquids. For example, the absorption coefficient (a) depends on the nature and contents of the liquid, which change according to disease and disease advancement, and liquid concentrations: both can be difficult to estimate clinically. For example, absorption coefficient differs from 0.05 to 6.9 cm⁻¹ for 0.9% saline and BSS (Balanced Salt Solution, respectively.

In addition, in UV lasers are often controlled to deliver multiple pulses. However, each pulse produces a certain amount of tissue ablation, thereby changing the distance between illuminating probe and the tissue and the contents and nature of the surrounding liquid. This results in a continually changing surgical environment.

One existing approach is illustrated schematically in FIG. 1 at 10, which shows an optical probe 12 for applying ultraviolet light 14 from a laser source (not shown) to a specimen 16—being an irradiated portion of a biological tissue in this example—in a liquid 18. Other portions of the tissue may not be in contact with liquid 18, but specimen 16 is regarded as in a liquid because liquid 18 and specimen 16 have an interface 20.

The forward or distal end 22 of optical probe 12 is tapered to a distal tip 24, which is also the exit from which the ultraviolet light 14 is emitted from optical probe 12. In use, there is a liquid layer 26 of the liquid 18 between distal tip 24 and specimen 16, and hence there is also an interface 28 between liquid 18 and distal tip 24, corresponding essentially to distal tip 24.

In use, ultraviolet light 14 is applied to specimen 16 in order to ablate specimen 16 (that is, remove surface portions of specimen 16). This leads, however, to the irradiation of liquid 18 in liquid layer 26 between distal tip 24 and specimen 16, causing changes to its composition, temperature and absorption coefficient. The ablation of specimen 16 also progressively increases the distance between the optical probe 12 and specimen 16, and the material removed by ablation further alters the composition of liquid 18 and hence its absorption coefficient.

Thus, liquid layer 26 between the optical probe 12 and the specimen 16 constitutes a complicated and unpredictable boundary, requiring consideration of (and potentially allowance for) micro-irradiation effects, laser biophysics, laser chemistry, laser biochemistry and probe-specimen distance, precluding a constant operational environment.

### SUMMARY OF THE INVENTION

According to a first broad aspect, the present invention provides an apparatus for irradiating a specimen (such as to ablate the specimen), the apparatus comprising:
an optical transmitter for transmitting light (such as ultraviolet light) from a laser source;
an optical probe with an optical exit, the optical probe configured to receive the light from the optical transmitter and to apply the light upon emission from said exit to the specimen;
a position detector (such as a force or other transducer or a detector) adapted to detect a position of the optical probe in a longitudinal (that is, z-axis or forward/reverse) direction and to output a signal indicative of said position or of a change in said position relative to a surface forward of the optical probe;
a drive coupled to the optical probe and adapted to controllably adjust a position of said optical probe in the longitudinal direction; and
a feedback controller adapted to receive the signal from said position detector (whether subsequently processed or not) and to control said drive to control said position to keep the optical probe at substantially a constant position relative to the surface forward of the optical probe.

Generally, the optical probe is adapted to be located when in use with the exit in contact with the specimen, in which case the material forward of the optical probe is the specimen.

Thus, the distance between optical probe and the surface (such as the specimen) affects, for example, ablation, so is thus advantageously controlled according to this aspect to be substantially constant. The present invention maintains the distance as effectively zero, which both is simpler to maintain and minimizes the effects of liquid—if used in a liquid environment—between probe and specimen (in those embodiments in which the surface is the specimen). It is expected that, although some liquid may be trapped between the probe and specimen, it will i) be minimal, and ii) be promptly evaporated during use, further reducing its quantity.

Thus, a generally gentle contact can be maintained between tip and specimen.

Although the apparatus is envisaged as principally for use for ablation, it could alternatively be incorporated into a fiberoptic laser endoscope and used to irradiate, for example, tumors (such as of the trachea, oesophagus or stomach). Such an endoscope typically comprises separate optical channels—terminating in the endoscope head—for imaging and specimen irradiation (according to this invention), respectively.

The optical probe could be, for example, solid or capillary, but is typically in the form of an optical fiber or an optical fiber bundle of optical fibers, such that the exit comprises the exit tip of the optical fiber or the exit tips of the optical fibers of the optical fiber bundle, respectively.

In one embodiment, the exit is at a distal tip of the optical probe and configured to emit the light in the longitudinal direction.

In one embodiment, the position detector comprises a force transducer coupled to the optical probe, wherein the force transducer is arranged to output a signal indicative of a force or a change in force between the optical probe and the surface, the feedback controller is adapted to output to the drive a control signal determined from the signal and the drive is adapted to receive the output signal and to control the position so as to maintain a substantially constant force between the optical probe and the surface (such as the specimen).

In another embodiment, the position detector comprises a probe adjacent to or coupled to the optical probe and having a force transducer, wherein the probe is arranged to contact the surface, in use, and output a signal indicative of a force or a change in force between the probe and the surface, the feedback controller is adapted to output to the drive a control signal determined from the signal and the drive is adapted to receive the output signal and to control the position so as to maintain a substantially constant force between the optical probe and the surface.

The apparatus may include a laser source for supplying the laser light. In applications in which the light is ultraviolet light, the laser source may comprise an ultraviolet laser source, or an infrared laser source and a mechanism for converting an output of the infrared laser source into ultraviolet light.

In another particular embodiment, the exit is located to emit the light laterally from the optical probe so as to irradiate the specimen when located beside the optical probe. The probe may be adapted to direct the light to exit the exit by reflecting the light towards the exit (such as with a mirror located in the probe, which may operate conventionally or by total internal reflection).

According to a second broad aspect, the present invention provides an endoscope comprising the apparatus described above.

According to a third broad aspect, the present invention provides an ablation apparatus comprising the apparatus described above.

According to a fourth broad aspect, the present invention provides a method of irradiating a specimen (such as to ablate the specimen), the method comprising:
locating an optical probe having an exit with the exit in contact with the specimen;
transmitting light (such as ultraviolet light) from a laser source to the optical probe; and
applying the light upon emission from the exit to the specimen;
detecting a position of the optical probe in a longitudinal direction with a position detector;
outputting from the position detector a signal indicative of the position or of a change in the position relative to a surface forward of the optical probe; and
controlling a drive coupled to the optical probe to control the position to keep the optical probe at substantially a constant position relative to the surface forward of the optical probe.

In one embodiment, the method includes driving the optical probe to maintain a position of the exit against the surface.

The method may include employing a feedback controller to control the drive according to the signal.

In another particular embodiment, the method includes emitting the light laterally from the optical probe and thereby irradiating the specimen located beside the optical probe. The method may include directing the light to exit the exit by reflecting the light towards the exit (such as with a mirror located in the probe, which may operate conventionally or by total internal reflection).

According to a fifth broad aspect, the present invention provides a method of ablating a specimen, comprising the method described above.

It should be noted that any of the various features of each of the above aspects of the invention, and of the various features of the embodiments described below, can be combined as suitable and desired.

### BRIEF DESCRIPTION OF THE DRAWING

In order that the invention may be more clearly ascertained, embodiments will now be described, by way of example, with reference to the accompanying drawing, in which:

FIG. 1 is a schematic view of an optical probe for applying ultraviolet light to a specimen according to the background art;

FIG. 2 is a schematic view of a laser ablation system according to an embodiment of the present invention;

FIG. 3 is a schematic view of the optical probe for applying ultraviolet light to a specimen of the system of FIG. 2;

FIG. 4 is a schematic view of the optical probe for applying ultraviolet light to a specimen of the system of FIG. 2;

FIGS. 5A and 5B are schematic views of the optical probe of FIG. 4 in use;

FIGS. 6A to 6C are schematic views of optical probes according to other embodiments of the present invention, for use in variants of the system of FIG. 2.

### DETAILED DESCRIPTION

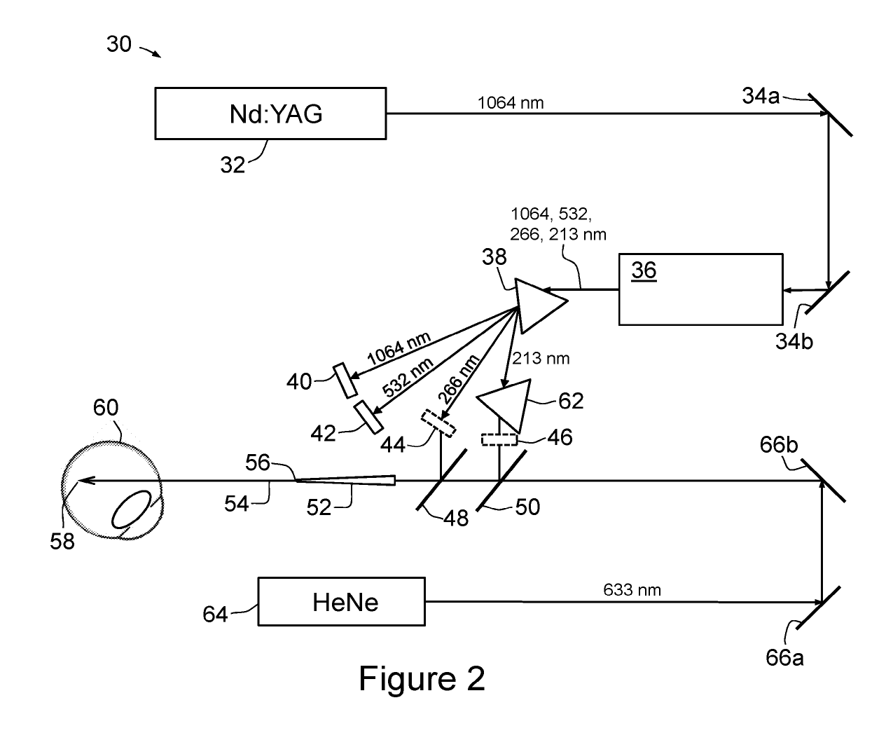

FIG. 2 is a schematic view of a laser ablation system according to an embodiment of the present invention. System 30 includes a Nd:YAG infrared laser source 32 that emits infrared light at 1064 nm. In the exemplary application described herein, for ablating a lesion, Nd:YAG infrared laser source 32 is controlled to deliver 1 to 100 pulses of light, each of 0.4-0.7 J/cm² and 4-6 ns duration, a pulse repetition rate of 10 Hz, a beam diameter of 6 mm, and a beam divergence of 0.6 mrad.

System 30 also includes a pair of mirrors 34a,34b that reflect the infrared light into a harmonic generator 36 that emits the infrared light as well as light at harmonic wavelengths 532 nm, 266 nm and 213 nm. Harmonic generator 36 comprises BBO crystals for generation of the second harmonic and CLBO crystals for generation of the fourth (266 nm) and fifth (213 nm) harmonics.

System 30 includes a dispersing prism 38 that receives the light emitted by the harmonic generator 36 and emits it dispersed according to wavelength, and first and second beam blocks 40 and 42 located to receive and block from further transmission the 1064 nm and 532 nm wavelength beams of light.

System 30 also includes moveable third and fourth beam blocks 44 and 46, and partially reflective mirrors 48 and 50. Third and fourth beam blocks 44 and 46 are locatable respectively in the optical paths of the 266 nm and 213 nm wavelength beams of light. A drive mechanism (not shown) allows third and fourth beam blocks 44 and 46 separately to be controlled to selectively pass or block each of these beams of light, and—when passed—these 266 nm and 213 nm wavelength beams impinge partially reflective mirrors 48 and 50, respectively.

Light at 266 nm and 213 nm closely matches absorption peaks of proteins in specimens of the type described below, but in other applications different wavelengths may be preferable and hence employed as necessary and suitable.

System 30 includes a hollow glass taper 52 for concentrating the beam, towards the larger (or entrance) end of which partially reflective mirrors 48 and 50 direct the reflected component of the 266 nm and 213 nm wavelength beam(s). Taper 52 is coupled at its distal or narrow end to the proximal end of an optical probe 54 (comprising an optical fiber, as described below) and thus launches the beam into the proximal end 56 of optical probe 54. The distal end of optical probe 54 is locatable against a specimen (in this example, an intraocular specimen, such as a portion 58 of is the retina of an eyeball 60).

It should be noted that, in system 30 (and other embodiments of the present invention) light may be transmitted by any suitable mechanism or medium. For example, some or all of the optical paths referred to above or shown in FIG. 2 may comprise free space, an optical transmitter such as an optical fiber or fiber bundle, or any suitable combination of these.

Thus, system 30 can be employed to irradiate and ablate specimen 58 with an ablating beam of wavelength 266 nm or 213 nm, or with components of wavelength 266 nm and of wavelength 213 nm.

System 30 includes a rotatable prism 62 located in the optical path between dispersing prism 38 and partially reflective mirror 50, which is rotatably adjustable so that the path of the 213 nm beam can be finely adjusted.

System 30 also includes a second laser source in the form of HeNe laser source 64, which emits visible light with a wavelength of 633 nm. Additional mirror pair 66a, 66b direct light from HeNe laser source 64 through partially reflective mirrors 48 and 50 (and hence into the same optical path as that of the ablating light) onto the specimen 58. This visible light allows, in effect, the visualisation of the location of incidence of the ablating beam (which, being in the ultraviolet, is invisible to the naked eye).

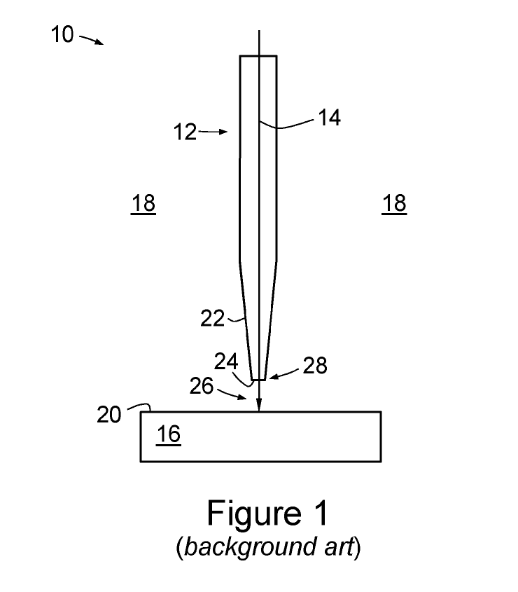

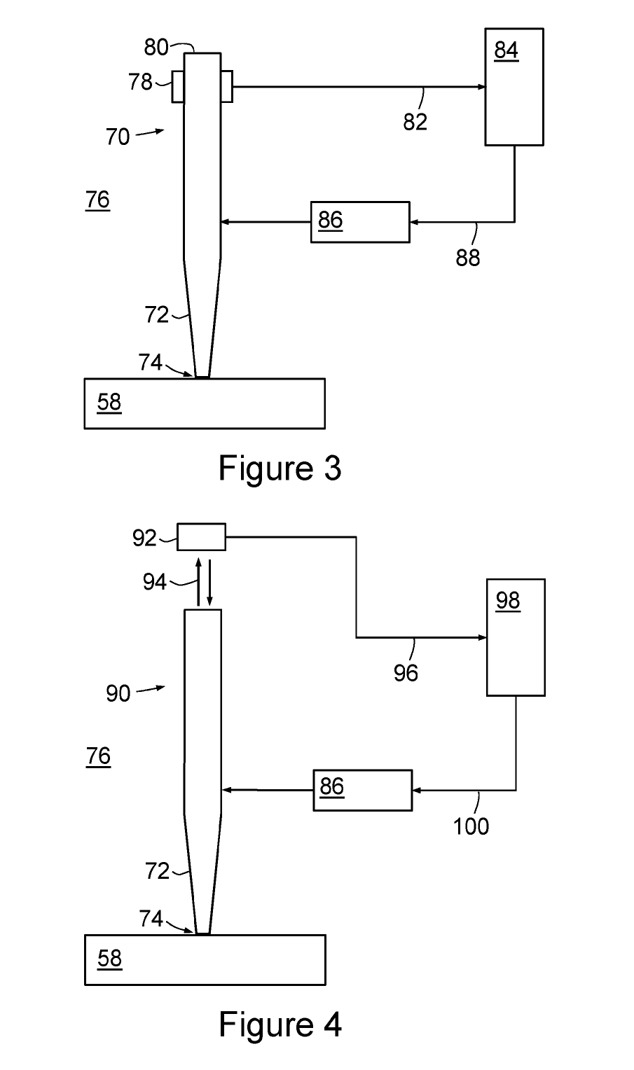

FIG. 3 is a schematic view of the optical probe of system 3 of FIG. 2, shown generally at 70, for applying ultraviolet light to specimen 58. Optical probe 70 is comparable to optical probe 12 of FIG. 1, and comprises an optical fiber of 800 mm length and 200 μm core diameter with a tapered forward or distal end 72 that is tapered to a distal tip 74 with a 60 μm diameter core. This core is also the exit from which ablating ultraviolet light and visualizing visible light are emitted from optical probe 70. In use, distal tip 74 is immersed in a surrounding liquid 76 and located in contact with specimen 58.

In use, distal tip 74 is located against specimen 58 (as is described in greater detail below). In use, optical probe 70 ablates a hole in the specimen of approximately 60 μm diameter, and from 40 to 400 μm depth depending on whether the optical probe 70 is not advanced or is advanced, respectably, between pulses.

Referring again to FIG. 3, system 30 includes a feedback control mechanism that includes a transducer 78 in the form of a force transducer, coupled to the optical probe 70 towards the proximal end 80 of optical probe 70 and hence, in use in this example, located outside eyeball 60. Transducer 78 is essentially responsive to longitudinal movement in the position of optical probe 70, and configured to output a signal indicative of a force, or change in force, caused by such longitudinal movement. The feedback control mechanism of system 30 also includes a feedback controller 84 and a drive 86 coupled to optical probe 70 for moving optical probe 70 in a longitudinal direction. Output signal 82 is transmitted to feedback controller 84, which generates a control signal 88 for drive 86 adapted to control drive 86 to drive optical probe 70 so as to restore the force (or eliminate the change in force) detected by transducer 78.

Thus, once optical probe 70 has been located as desired against the specimen 58, such that distal tip 74 exerts a gentle force against specimen 58, this feedback control mechanism—comprising transducer 78, feedback controller 84 and drive 86—is activated and holds distal tip 74 against the specimen 58 so that the original gentle force is maintained.

FIG. 4 is a schematic view of the optical probe 90 for use in a variation of system 30 to apply ultraviolet light to specimen 58, according another embodiment of the present invention. Optical probe 90 is identical in many respects with optical probe 70 of FIG. 3, and like reference numerals have been sued to identify like features. However, in this embodiment optical probe 90 is provided with a feedback control mechanism having a transducer 92 in the form of an optical sensor. Transducer 92 is located to receive a portion 94 of the light transmitted from specimen 58, hence providing an output signal 96 that is a measure of the level of contact between distal tip 74 and specimen 58 (as removal of distal tip 74 from specimen 58 will reduce the intensity of return light captured by distal tip 74 and transmitted to transducer 92). Feedback controller 98 of this embodiment uses this signal 96 to generate a control signal 100 for drive 86 adapted to control drive 86 to drive optical probe 70 so as to restore the intensity of return light detected by transducer 92. Thus, in this embodiment the position of the distal tip 74 in gentle contact with specimen 58 is preserved, by a feedback control mechanism comprising transducer 92, feedback controller 98 and drive 86.

It will also be appreciated that the feedback control mechanism of FIGS. 3 and 4 could, in another embodiment, both be employed in the one system. This would allow the use of feedback based on two simultaneous measures of the position of the distal tip.

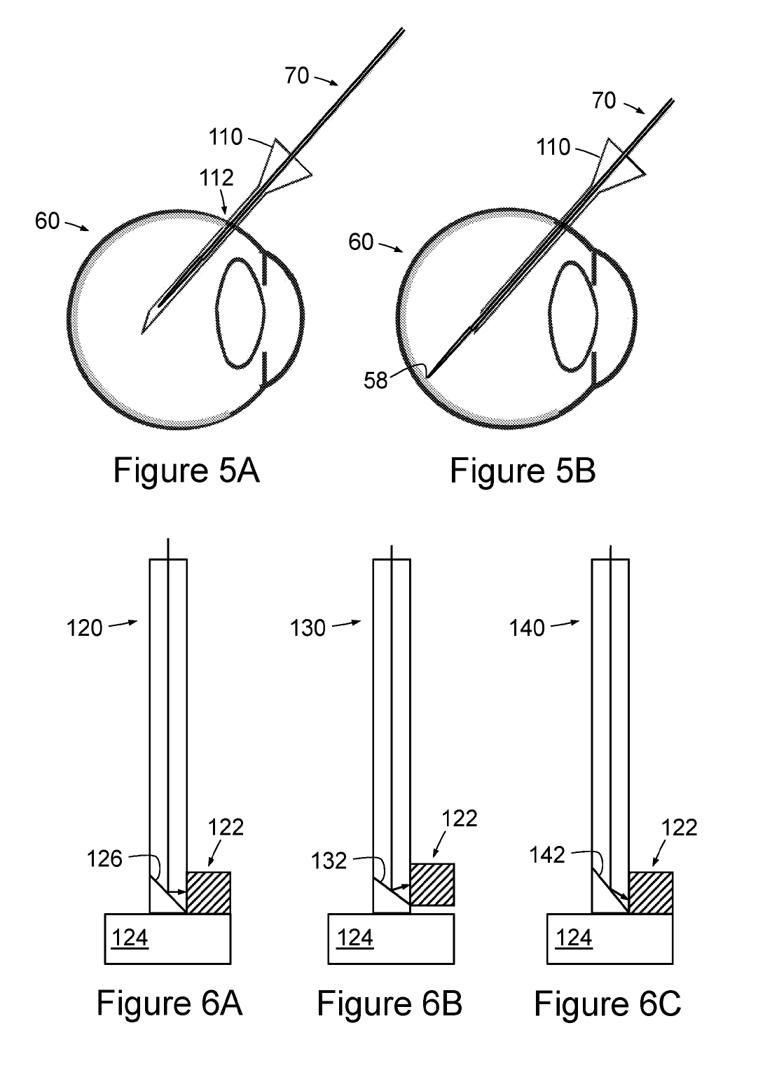

FIGS. 5A and 5B illustrate the placing of optical probe 70,90 into the appropriate location for ablation of specimen 58, which—as described above—comprises in this example a portion of the retina of an eyeball 60. Referring to FIG. 5A, the leading or distal portion of optical probe 70 is located inside a 25G needle 110, which is used to penetrate the wall 112 of eyeball 60 through the pars plana or other location, according to target specimen/tissue.

Referring to FIG. 5B, optical probe 70 is then advanced inside eyeball 60 until in gentle contact with and just touching specimen 58. This contact can be judged by visualisation under an operating microscope or endoscope. Alternatively, the degree of contact with specimen 58 can be assessed by monitoring an output signal from transducer 78 or 92 (according to the embodiment) or from feedback controller 84 or 98, to ensure that distal tip 74 tip just touches the specimen 58. The feedback control mechanism is then employed to maintain the longitudinal position of optical probe 70 as described above. The position of optical probe 70 in other directions is maintained by conventional techniques.

FIGS. 6A to 6C are schematic views of optical probes according to other embodiments of the present invention, for use in variants of the system of FIG. 2 with specimens that are laterally adjacent the distal tip of the respective optical probe.

These embodiments would typically be preferred when the specimen or target tissue is adjacent to normal tissue, and it is desired to protect the normal tissue.

FIG. 6A is a schematic view of an optical probe 120 according to an embodiment of the present invention in use with a specimen 122 that is itself adjacent to normal tissue 124. In this embodiment, optical probe 120 is not tapered, but instead includes a 45° mirror 126 at the distal end of optical probe 120 that deflects incoming light 90° so that it is emitted from an exit into a specimen laterally adjacent optical probe 120. The ablating irradiation is therefore not directed towards the normal tissue 124, which in the configuration of optical probe 70 of FIG. 3 might pass through the specimen 122 and into normal tissue 124 below (in this view) specimen 122.

Mirror 126 may be provided in any suitable way, such as by providing optical probe 120 with an oblique distal tip with a silvered surface, or an internal, mirrored surface.

FIG. 6B is a schematic view of an optical probe 130 according to another embodiment. Optical probe 130 is comparable to optical probe 120, except that—instead of a 45° mirror—optical probe 130 has a mirror 132 that deflects light through an obtuse angle and hence somewhat upwardly (in this view), such as by 100° or 110°. Thus, specimen 122 may be irradiated even though somewhat further above the normal tissue 124 than in the example shown in FIG. 6A.

FIG. 6C is a schematic view of an optical probe 140 according to still another embodiment. Optical probe 140 is again comparable to optical probe 120, except that—instead of a 45° mirror—optical probe 140 has a mirror 142 that deflects light through an acute angle and hence somewhat downwardly (in this view), such as by 70° or 80°. Thus, specimen 122 may be irradiated even though closer to normal tissue 124 than in the example shown in FIG. 6A.

In each of the embodiments of FIGS. 6A to 6C, the feedback control mechanism comprises a force transducer (as described above by reference to FIG. 3), and controls the respective optical probes to maintain position relative to normal tissue 124, and hence relative to specimen 122.

Modifications within the scope of the invention may be readily effected by those skilled in the art. It is to be understood, therefore, that this invention is not limited to the particular embodiments described by way of example hereinabove.

In the claims that follow and in the preceding description of the invention, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments of the invention.

Further, any reference herein to prior art is not intended to imply that such prior art forms or formed a part of the common general knowledge in Australia or any other country.

1-16. (canceled)

17. An apparatus for irradiating an intraocular specimen comprising:
an optical transmitter for supplying ultraviolet light by transmitting light from a laser source;
an optical probe with an optical exit, the optical probe configured to receive the ultraviolet light from the optical transmitter and to apply the ultraviolet light upon emission from the exit to the specimen;
a position detector adapted to detect a position of the optical probe in a longitudinal direction and to output a signal indicative of the position or of a change in the position relative to a surface forward of the optical probe;
a drive coupled to the optical probe and adapted to controllably adjust a position of the optical probe in the longitudinal direction; and
a feedback controller adapted to receive the signal from the position detector and to control the drive to control the position to keep the optical probe at substantially a constant position relative to the surface forward of the optical probe.

18. The apparatus of claim 17, wherein the optical probe comprises an optical fiber or an optical fiber bundle.

19. The apparatus of claim 17, wherein the exit is at a distal tip of the optical probe.

20. The apparatus of claim 17, wherein the position detector comprises a force transducer coupled to the optical probe and arranged to output a signal indicative of a force or a change in force between the optical probe and the surface, the feedback controller is adapted to output to the drive a control signal determined from the signal and the drive is adapted to receive the output signal and to control the position so as to maintain a substantially constant force between the optical probe and the surface.

21. The apparatus of claim 17, wherein the position detector comprises a probe adjacent to or coupled to the optical probe and having a force transducer, wherein the probe is arranged to contact the surface, in use, and output a signal indicative of a force or a change in force between the probe and the surface, the feedback controller is adapted to output to the drive a control signal determined from the signal and the drive is adapted to receive the output signal and to control the position so as to maintain a substantially constant force between the optical probe and the surface.

22. The apparatus of claim 17, wherein the laser source comprises an infrared laser source and a mechanism for converting an output of the infrared laser source into ultraviolet light.

23. The apparatus of claim 17, wherein the exit is located to emit the light laterally from the optical probe so as to irradiate the specimen when located beside the optical probe.

24. The apparatus of claim 17, wherein the feedback controller drives the optical probe to maintain a distal tip of the optical probe against the surface.

25. An ablation apparatus comprising the apparatus of claim 17.

26. A method of irradiating an intraocular specimen comprising:
transmitting ultraviolet light from a laser source to an exit of an optical probe;
applying the ultraviolet light upon emission from the exit to the specimen;
detecting a position of the optical probe in a longitudinal direction with a position detector;
outputting from the position detector a signal indicative of the position or of a change in the position relative to a surface forward of the optical probe; and
controlling a drive coupled to the optical probe based on the signal to control the position to keep the optical probe at substantially a constant position relative to the surface forward of the optical probe.

27. The method of claim 26, wherein the optical probe comprises a distal tip and the method further comprises driving the optical probe to maintain the distal tip against the surface.

28. The method of claim 26, further comprising employing a feedback controller to control the drive according to the signal.

29. The method of claim 26, further comprising emitting the light laterally from the optical probe and thereby irradiating the specimen located beside the optical probe.

30. The method of claim 29, further comprising directing the light to exit the exit by reflecting the light towards the exit.

31. The method of claim 26, wherein transmitting ultraviolet light from a laser source comprises emitting light from an infrared laser source and converting an output of the infrared laser source to ultraviolet light.

32. A method of ablating a specimen, comprising the method claimed in claim 26.
